(19) United States
(12) Patent Application Publication
(10) Pub. No.: US 2017/0202487 A1
(43) Pub. Date: Jul. 20, 2017

(54) DIAGNOSTIC TOOL FOR MEASURING OSSICULAR CHAIN COMPLIANCE

(71) Applicant: THE REGENTS OF THE UNIVERSITY OF COLORADO, A BODY CORPORATE, Denver, CO (US)

(72) Inventor: Justin CASEY, Denver, CO (US)

(73) Assignee: The Regents of the University of Colorado, a Body Corporate, Denver, CO (US)

(21) Appl. No.: 15/302,142

(22) PCT Filed: May 4, 2015
(86) PCT No.: PCT/US2015/029099

§ 371 Date: Oct. 5, 2016

Related U.S. Application Data
(60) Provisional application No. 61/987,915, filed on May 2, 2014.

(51) Int. Cl. A61B 5/12; A61B 17/34; A61B 17/24; A61B 5/11; A61B 5/00

(52) U.S. Cl. CPC A61B 5/125; A61B 5/1126; A61B 5/6847; A61B 17/24; A61B 17/3423; A61B 2017/345

(57) ABSTRACT

The disclosure provides systems and methods of use pertaining to precise measurements of middle ear mechanics to aid physicians in the determination of pre-operative, intraoperative, and post-repair stiffness of the ossicular chain to assist in the diagnosis and treatment and/or repair of all types of conductive hearing loss. One embodiment provides a handheld diagnostic tool having a load cell operably coupled between a linear actuator and a rigid probe configured to rest against the ossicular chain. When the actuator displaces the load cell and the rigid probe a predetermined distance in an oscillating manner, the load cell measures a force required to displace the ossicular chain a corresponding distance. Force and displacement data is sent to a processing element, which calculates a stiffness of the ossicular chain and reports information regarding the stiffness to a display. Other embodiments are also disclosed.

### BACKGROUND

Over 12% of the U.S. population, or 38 million Americans, suffer from hearing loss. Although some can only be offered hearing aids or other imperfect stopgaps, many of these patients can be helped with surgical interventions. Conductive hearing loss is one of two broad categories of hearing loss, in which the structures of the external or middle ear are not transmitting and amplifying sound properly. This type of hearing loss can include anything from a hole or scarring of the ear drum, to fluid or scar tissue in the middle ear space, to fractures or metabolic bone disorders affecting the smallest bones in the body (the “ossicles” also known as the “ossicular chain”) that reside in the middle ear, typically each about the size of an uncooked grain of white rice.

Fortunately, the corrective surgery available for conductive hearing loss is relatively safe and has limited adverse effects. The reliability of diagnosis, however, is not as reassuring. Many aspects of hearing loss are difficult to diagnose, and can even rely on guessing and “eye-balling” the microscopic motions of the middle ear structures during surgery, structures that move on the order of 10-100 microns.

Current diagnostic methods for conductive hearing loss rely on an audiogram with tympanometry (a hearing test with air pressure to assess ear drum motion). This method gives the physician an idea of the motion of the ear drum and a vague idea of the type of hearing loss the patient is experiencing, but it does not provide an exact diagnosis. An actual “diagnosis” is currently typically obtained by performing a diagnostic surgical procedure known as a “middle ear exploration.”

The middle ear exploration involves incisions in or around the ear to access the area behind the eardrum, called the “middle ear space,” where the surgeon applies a fine metal rod to tap or palpate the ossicles. During palpation of these bones, the surgeon makes an assessment of the microscopic motion and stiffness of the chain of bones. This assessment is extremely subjective, and lab studies have suggested that the force required to move a healthy ossicular chain is as low as 20 Pascals, or ˜0.15 mmHg, while the displacement of the ossicles during normal vibrations is on the order of microns. Though these magnitudes of force and displacement are nearly manually undetectable, the surgeon's judgment with the naked eye while “tapping” the ossicles is currently used to determine whether or not to proceed with corrective surgery and what type of surgery to perform.

If this initial palpation of the ossicles during the middle ear exploration procedure results in an opinion that the ossicular chain is stiff, the surgeon may further explore the middle ear to inspect the integrity of the ossicles by looking for fractures, scar tissue, or other obstructive soft tissues impacting the function of the ossicular chain. If there is no visible problem with the ossicles, it is presumed that the chain “feels stiff” because of a disease called Otosclerosis. In a healthy state, the three bones forming the ossicular chain (the stapes, incus, and malleus bones), the joints between each bone, and the eardrum work together to help amplify sound that enters the ear canal. Otosclerosis causes the ossicular chain to undergo a type of boney remodeling that leads to stiffening of the ossicles, which can cause conductive hearing loss.

There is currently no lab or other test to positively diagnose otosclerosis, making the process a presumed diagnosis of exclusion. This presumed diagnosis generally results in the surgeon removing one or two of the ossicles for replacement with tiny titanium prosthesis.

Similar to other bones in the human body, each person exhibits slight differences in ossicle size. These variances introduce additional subjectivity into the surgery used to correct conductive hearing loss. To size the prosthesis, the surgeon typically uses a tiny measuring stick placed into the ear. Once the prosthesis is placed, the ossicle-prosthesis combination is again subjectively tapped to check for the stiffness of the repair before the surgical site is closed, and the patient is awakened and sent home. When the blood clot dissolves in a month or so, hearing is tested in clinic. If hearing improves, the surgeon assumes he was right and the correct diagnosis was otosclerosis. If hearing does not improve, further workup of the patient's hearing loss is pursued.

Both the middle ear exploration procedure used to diagnose otosclerosis and the surgery used to correct the problem are unacceptably subjective and prone to error. These trial and error procedures make up the current standard of care, and are performed in operating rooms around the country every day. Not only is there no perfect test in the clinic, but in the operating room surgeons are forced to make decisions based on subjective and non-quantitative judgments of microscopic forces and movements. These processes are fraught with inaccuracies and misdiagnoses and often lead to unnecessary surgeries. The human hand, no matter how precise or well trained, is simply not able to sense subtle motions or the very minute amount of force required to displace the ossicles. Additionally, surgeons face challenges in sizing appropriate prosthesis and are currently unable to assess the integrity of their repairs after placement of the prosthesis. Thus, there is a need for a quantitative measurement of the stiffness of the ossicular chain to assist in the determination of pre-operative, intra-operative, and post-repair stiffness of the ossicles for the diagnosis and treatment of all types of conductive hearing loss.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

One embodiment provides a diagnostic tool for measuring an intraoperative stiffness of an ossicular chain of a human ear. The tool includes a chassis. The chassis houses a load cell that is operatively coupled to a linear actuator and a processing element. The tool also includes a rigid probe protruding from the chassis from a proximal end to a distal end. The proximal end is operatively coupled to the load cell and the distal end is configured for placement against the ossicular chain of the human ear. When the diagnostic tool is activated, the linear actuator displaces the load cell and the rigid probe through a predetermined distance in an oscillating manner, the load cell measures a force of the distal end of the rigid probe against the ossicular chain, and the processing element receives a data signal relating to the predetermined distance and the force and calculates a stiffness of the ossicular chain.

Another embodiment provides a system for analyzing the compliance of an ossicular chain of a human ear. The system includes a diagnostic system having a load cell coupled between a linear actuator and a rigid probe, the rigid probe having a proximal end and a distal end adapted to abut the ossicular chain. When activated, the linear actuator oscillates the load cell through a predetermined distance, and the load cell measures a force exerted between the distal end of the rigid probe and the ossicular chain. The system also includes a processing element coupled with the load cell. The processing element includes logic instructions configured to analyze displacement and force data from the diagnostic system and calculate a stiffness of the ossicular chain. The system additionally includes a handheld chassis housing the load cell, the linear actuator, and the processing element, where the proximal end of the rigid probe protrudes from the handheld chassis.

Yet another embodiment provides a method of measuring an intraoperative stiffness of an ossicular chain of a human ear using an operative speculum and a handheld ossicle-measurement tool. The operative speculum has an instrument port configured to align with the ossicular chain when the speculum is placed within an ear canal, and the handheld ossicle-measurement tool has a load cell operatively coupled between a rigid probe and a linear actuator, where the load cell and the linear actuator are in communication with a processing element and a display. The method includes placing the speculum within the ear canal such that the speculum provides visual access to the ossicular chain, introducing the rigid probe of the handheld ossicle-measurement tool into the instrument port of the operative speculum such that a distal end of the probe contacts the ossicular chain, and activating the ossicle-measurement tool such that (1) the linear actuator displaces the distal end of the rigid probe a predetermined distance against the ossicular chain, (2) the load cell measures a force of the rigid probe against the ossicular chain required to achieve movement of the predetermined distance, and (3) the processing element receives displacement and force data and calculates a stiffness of the ossicular chain.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 illustrates a functional diagram of one embodiment of a diagnostic tool for measuring the functionality of the ossicular chain of the human ear;

FIG. 2 illustrates an exploded view of a diagnostic system incorporated within the diagnostic tool of FIG. 1;

FIG. 3 illustrates perspective views of numerous exemplary probe tips for a rigid probe of the diagnostic system of FIG. 2;

FIG. 4 illustrates a perspective view of another embodiment of a diagnostic tool having an angled probe for measuring the functionality of the ossicular chain of the human ear;

FIG. 5 illustrates a prospective view of one embodiment of an operative speculum for use with the diagnostic tool of FIG. 1;

FIG. 6 illustrates a top plan view of the operative speculum of FIG. 5;

FIG. 7 illustrates a front plan view of the operative speculum of FIG. 5;

FIG. 8 illustrates a side plan view of the operative speculum of FIG. 5;

FIG. 9 illustrates a prospective view of the diagnostic tool of FIG. 1 inserted into an insertion port of the operative speculum of FIGS. 5-8;

FIG. 10 illustrates a perspective view of the diagnostic tool of FIG. 1 engaged with the operative speculum of FIGS. 5-8 and inserted into an ear canal; and

FIG. 11 shows a flow chart detailing an exemplary method of use for the diagnostic tool of FIG. 1 and the speculum of FIGS. 5-8.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

Various embodiments of the systems and methods described herein relate to a diagnostic tool for assessing or measuring compliance of the ossicular chain within the human ear. The diagnostic tool discussed below provides an elegant, handheld solution to perform extremely precise measurements of middle ear mechanics, including measurements relating to the ossicular chain, in order to aid physicians in the determination of pre-operative, intraoperative, and post-repair stiffness of the ossicles to assist in the diagnosis and treatment and/or repair of all types of conductive hearing loss.

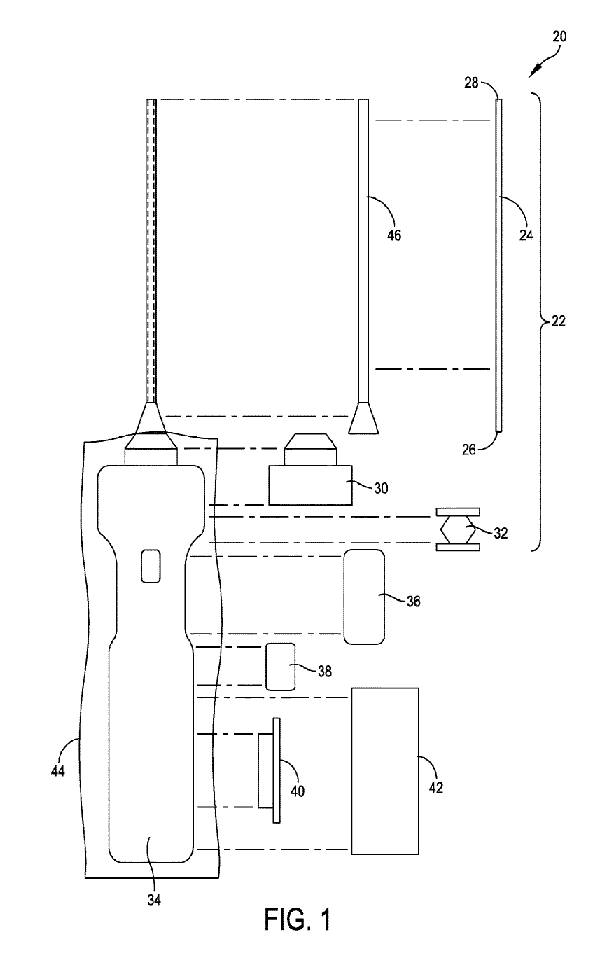

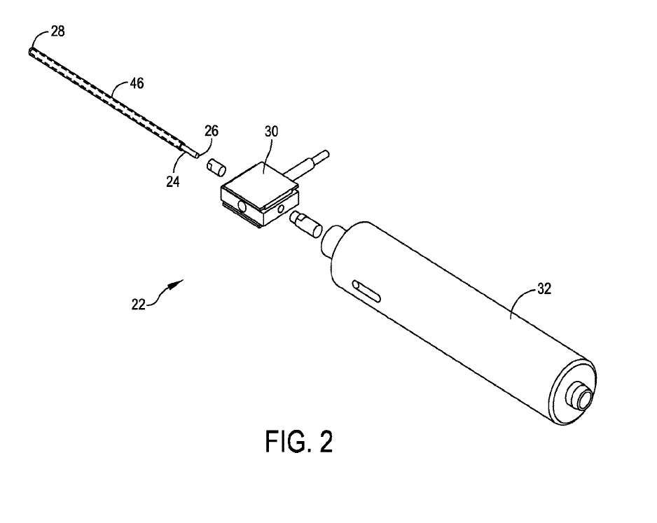

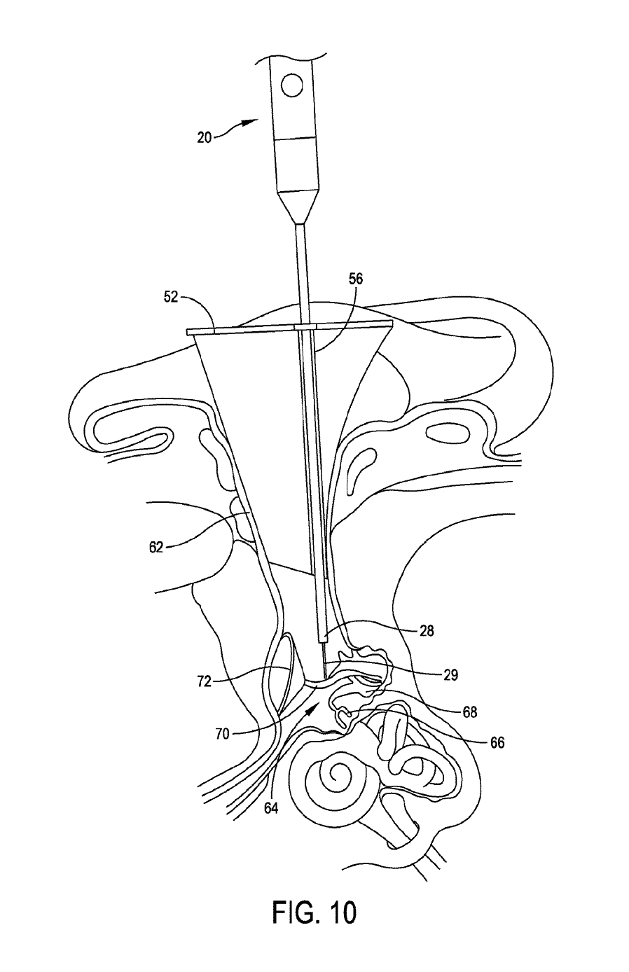

FIG. 1 illustrates a functional diagram of one embodiment of a handheld diagnostic tool 20, or otosclerosis-pen (“oto-pen”), for measuring an intraoperative stiffness of the ossicular chain of the human ear. In this embodiment, diagnostic tool 20 may include a diagnostic system 22, detailed in the exploded view of FIG. 2. Diagnostic system 22 includes a rigid probe 24 having a proximal end 26 and a distal end 28. Proximal end 26 of rigid probe 24 may be operably coupled to a sensitive load cell 30 (e.g., a micro strain gauge), capable of sensing minimal forces on the order of 0.1 N applied to distal end 28 of rigid probe 24, which may be adapted for placement against the ossicular chain of the human ear, as discussed in greater detail below and specifically in relation to FIG. 10.

The interconnected probe 24 and load cell 30 may, in turn, be coupled to a small linear oscillator or actuator 32. Linear actuator 32 may be configured to displace load cell 30 and rigid probe 24 a predetermined distance in an oscillatory or piston-like fashion against the ossicular chain. This motion may occur in a single cycle or in multiple oscillatory cycles. To accommodate the miniscule incremental movements of the ossicular chain, linear actuator 32 may take any appropriate size, shape, type, and/or configuration capable of achieving an extremely small predetermined oscillatory distance, on the order of 1-50 microns.

Once actuator 32 has initiated motion, which translates through to the ossicular chain, load cell 30 may continuously measure the force of probe 24 against the ossicular chain or eardrum and send these values to a processing element, discussed below, for processing force and/or stiffness calculations. Embodiments of load cell 30 and linear actuator 32 may be separate units, or they may be combined as a single functioning unit.

Rigid probe 24 may be formed of a stiff, brittle metal such as, for example, titanium or stainless steel, to minimize the risk of bending and/or deformity during measurements, which could result in a loss of force transferred to load cell 30. Probe 24 may vary in diameter from 0.15 mm to 3 mm, depending on patient physiology, application, and/or surgical environment. A diameter of 1 mm or less provides optimal maneuverability through the ear canal and within the middle ear.

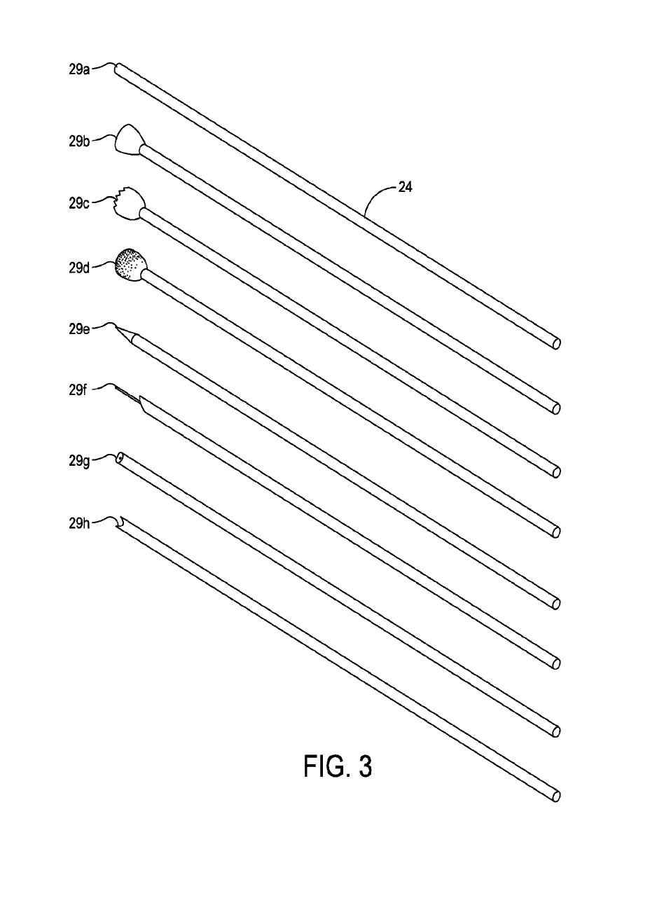

Distal end 28 of rigid probe 24 may terminate at a tip 29 having a variety of shapes adapted to accommodate varying surgical and ossicular-chain scenarios. FIG. 3 shows perspective views of a number of exemplary tips 29a-h for probe 24, including a spherical tip 29a, a smooth flattened tip 29b, a flattened tip with fine ridges 29c for better friction and less slippage against the ossicles, a flattened tip with a roughened or gritty surface 29d, a pointed tip 29e, a needle tip 29f, a concave tip 29g, and a u-shaped tip 29h.

As shown in FIG. 1, diagnostic assembly 22 may be incorporated into the larger structure of diagnostic tool 20 via a durable handheld chassis 34. In this embodiment, chassis 34 may receive proximal end 26 of rigid probe 24 (via, for example, but not limited to, either a threaded or a snap fit) and also house load cell 30 and linear actuator 32, as well as a processing element 36, an analog/digital converter 38, a display 40, and a power source 42 such as a battery or an AC adapter.

Chassis 34 may be formed of any appropriate material, such as, for example, metal or plastic that renders chassis 34 reusable between procedures. Chassis 34 may also have any appropriate size and/or shape to achieve an ergonomic design that fits comfortably within a surgeon's hand and that may be angled in a manner that prevents the surgeon's hand and/or diagnostic tool 20 from blocking the surgical view of the middle ear during a procedure. Chassis 34 may also be coated with an anti-slip material such as silicone, rubber, nitrile, or another polymer to enhance the surgeon's grip on diagnostic tool 20. In addition, ridges and/or divots may be added for ergonomic comfort.

To protect chassis 34 and to facilitate the reusability of tool 20 in a sterile surgical environment, chassis 34 may be used in conjunction with a sterile cover 44 for intraoperative use. Cover 44 may envelop the entirety of chassis 34 and, in one embodiment, may be formed of a disposable, transparent plastic (e.g., a flexible plastic bag). In another embodiment, chassis 34 may be formed of reusable metal or plastic forming an exoskeleton-type housing that may be sterilized in a high temperature auto-clave between uses. One embodiment of cover 44 may feature ridges or grooves to enhance gripping friction.

Like cover 44 for chassis 34, rigid probe 24 may be enveloped within a close-fitting probe sheath 46, shown in FIGS. 1-2. In one embodiment, sheath 46 may slip over probe 24 in a manner that connects into, and is removable from, chassis 34. Sheath 46 serves to protect probe 24 from external forces exerted during manipulation through the ear canal and middle ear. Such external forces on probe 24 could lead to inaccurate load-cell readings. Beyond a physical barrier, sheath 46 may also include a biocompatible lubricant to reduce frictional forces on probe 24. Both probe 24 and sheath 46 may be designed for either single or multiple-use disposability, or they may be reusable and safe for auto-clave sterilization.

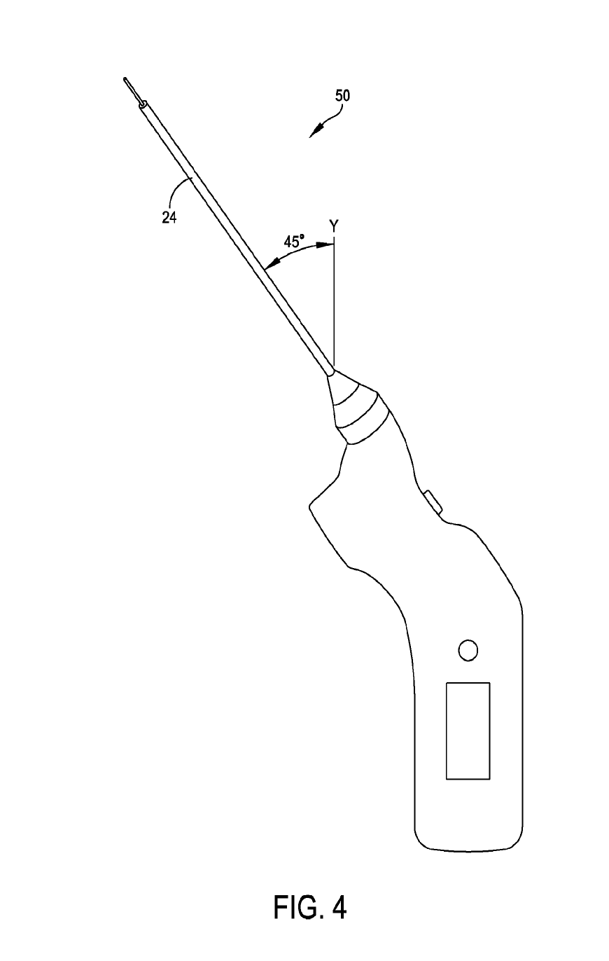

While FIG. 1 depicts probe 24 as protruding from chassis 34 in a vertical, linear fashion, other embodiments of diagnostic tool 20 may be configured such that probe 24 is coupled to load cell 30 and linear actuator 32 at an angle ranging from 15 to 90 degrees as appropriate to prevent blockage of the surgeon's view, either with the naked eye or through an operating microscope present in the operating theater during a procedure. To demonstrate one example, FIG. 4 shows an alternate embodiment of a diagnostic tool 50, in which probe 24 is angled at approximately 45 degrees from a vertical axis, Y.

Processing element 36 housed within chassis 34 may include executable logic configured to analyze and evaluate force and displacement data sent from load cell 30 and/or actuator 32. As a result of this analysis, processing element 36 may provide a number of useful results regarding a status of the ossicles, including, for example, real time force values exhibited during a single or multiple cycles of movement of the ossicles, a maximum force measured during a single or multiple cycles of movement of the ossicles, an average force measured over multiple cycles, and/or a force-displacement curve based on a complete movement cycle. To ensure measurement accuracy, processing element 36 may be programmed to move probe 24 until it begins sensing a force (i.e., until it contacts the ossicular chain) before beginning true force measurements. Alternatively, a chime may sound or a light may flash on display 40 to alert the surgeon when probe 24 has contacted the ossicle and when force begins to be measured.

Processing element 36 may also calculate a stiffness of the ossicular chain through the equation: k=F/δ, where k is the stiffness, F is the measured force, and δ is the predetermined displacement (or the known oscillatory distance) of probe 24 against the ossicular chain. This patient-specific calculated stiffness may be compared to normative data collected in studies on heathy ears to yield a simple assessment of “stiff,” “loose,” or “normal” in relation to the patient in question.

Upon completion of the analysis, processing element 36 may then send results calculated during the analysis to analog/digital converter 38 and on to display 40. Display 40 may be a liquid-crystal display (an “LCD”) or any other appropriate type of commercially available display. As an alternative to written results, display 40 may provide a color response that coordinates with particular diagnoses relating to the ossicular chain (e.g., green equates to “normal,” yellow equates to “loose,” and equates to equals “stiff”), allowing the surgeon to assess the analysis by seeing a color in his or her peripheral vision without removing his attention from the surgical field.

Diagnostic tool 20 may be positioned against the ossicular chain of the middle ear with the help of a support and positioning device. The support and positioning device may be configured to steady tool 20 for precise force and displacement measurements and to render tool 20 compatible with an operative microscope, an endoscope, other surgical instrumentation, and/or a surgeon's hand.

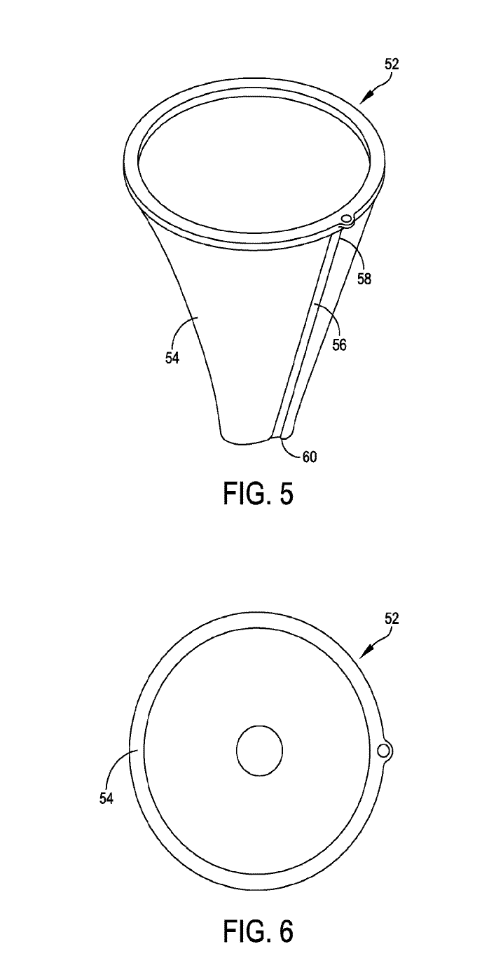

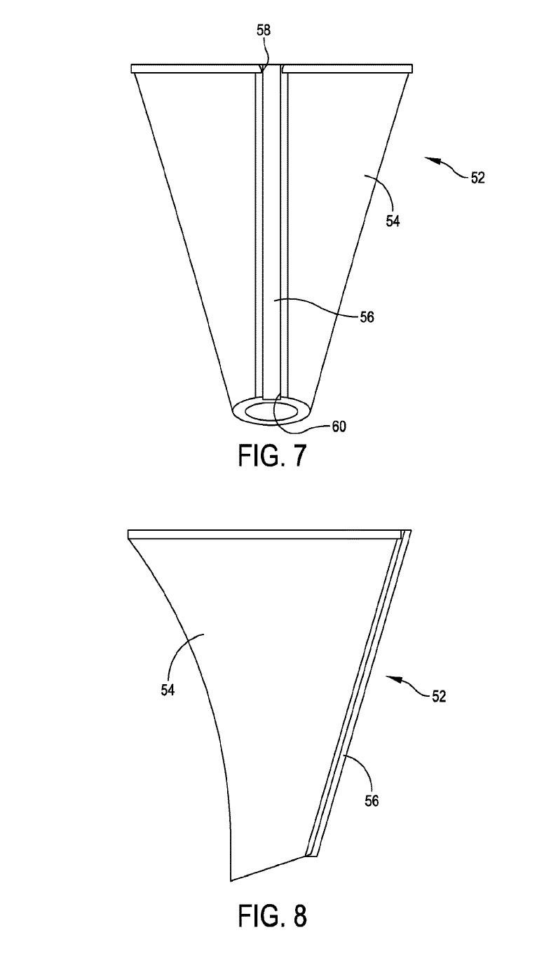

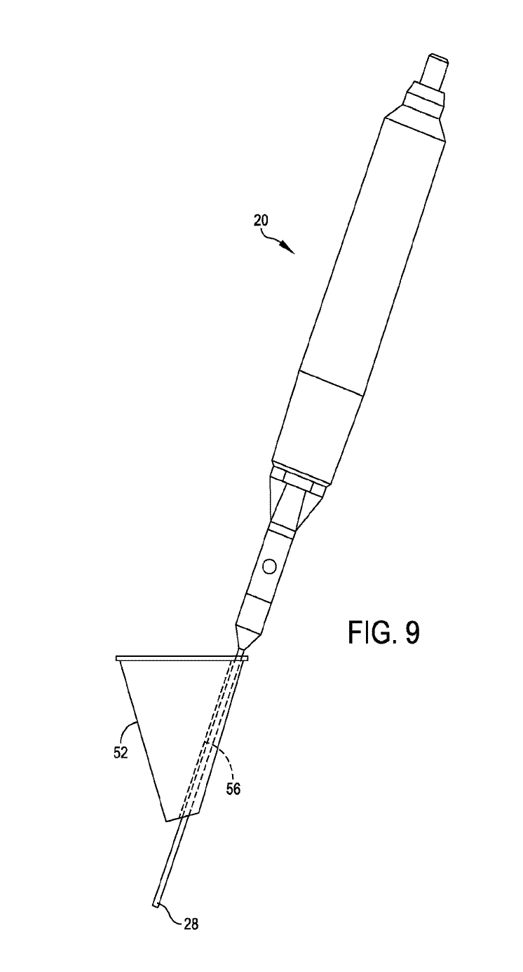

FIGS. 5-8 show respective perspective, top, front, and side views of one embodiment of a support and positioning device, which in an exemplary embodiment is an operative speculum 52, designed to support diagnostic tool 20 during an operative procedure, discussed above. In this embodiment, operative speculum 52 includes a tapered body 54 and an instrument port 56. Instrument port 56 may have a diameter sufficient to receive and retain probe 24 (either entirely enclosed or held within a semi-circular channel) when probe 24 is encompassed within probe sheath 46 as diagnostic tool 20 is being used in the middle ear. Instrument port 56 may have a top end 58 and a bottom end 60 and may be positioned within body 54 of speculum 52 or it may sit outside body 54. In one embodiment, bottom end 60 of port 56 may be tapered to an identical shape/diameter of probe sheath 46 such that rigid probe 24 and probe sheath 46 fit snuggly within port 56 and “lock” into port 56 at a maximally engaged position, shown in FIGS. 9 and 10. Exemplary embodiments of instrument port 56 may range from <1 mm to 4.2 mm. In operation, port 56 may steady tool 20 in the surgeon's hand by taking weight off of the surgeon's hand during measurements. Additionally, the close fit and locking functionality of instrument port 56 may overcome the challenges of supporting tool 20 with the naked human hand. These challenges include natural human tremors and the additive force of the surgeon's hand pressing tool 20 against the ossicular chain rather than allowing probe 24 to consistently and gently make contact with the ossicular chain.

To demonstrate, FIGS. 9 and 10 illustrate diagnostic tool 20 as supported at the maximally engaged position, discussed above, by one embodiment of operative speculum 52. As shown in FIG. 10, operative speculum 52 may be placed within an ear canal 62 such that it manipulates the soft tissues of ear canal 62 to provide the best line of sight for the surgeon to view the middle ear space while looking through an operative microscope (not shown). Once speculum 52 is placed, tool 20 may be inserted into instrument port 56 such that distal end 28/tip 29 as well as sheath 46 (not shown) of diagnostic tool 20 rest against an ossicular chain 64, which is composed of an interconnected stapes bone 66, an incus bone 68, and a malleus bone 70. In some instances, probe 24 may be brought into contact with an eardrum 72.

Operative speculum 52 may be formed from durable metal, such as titanium or stainless steel, and sterilized between procedures, or it may be formed of a disposable polymer. Polymer embodiments may be transparent to allow the surgeon to visually assess the ear canal and middle ear, as well as the placement of probe 24 within instrument port 56 of speculum 52. Alternatively, an embodiment of speculum 52 may be black with a matte surface, which serves to reduce the reflection of light from an operating microscope. Transparent polymer embodiments may also include fiber-optic veins to provide a light source powered by power source 42 of diagnostic tool 20. In another embodiment, operative speculum 52 may be formed of a thin, malleable metal that may be manipulated to best fit the patient's ear canal 62 during surgery. In this embodiment, while body 54 of speculum 52 may be malleable, instrument port 56 may remain rigid to properly support rigid probe 24/sheath 46. Embodiments of speculum 52 may be any appropriate diameter and/or length to compensate for varying ear canal sizes and lengths.

Another embodiment of a support and positioning device for diagnostic tool 20 may take the form of an articulating arm (not shown). The articulating arm may have several joints that allow for mobility and adjustment of the arm within numerous planes, allowing diagnostic tool 20 to best reach the middle ear. The articulating arm may be tightened or locked into position when probe 24 has made a preferred contact with the ossicular chain. A base of the articulating arm may attach to the operating table via a clamp (e.g., a Universal Rail Clamp) that attaches to the table's side-rail. An instrument end of the articulating arm may clamp to chassis 34 of diagnostic tool 20 or may be screwed or otherwise fastened directly into an embodiment of chassis 34.

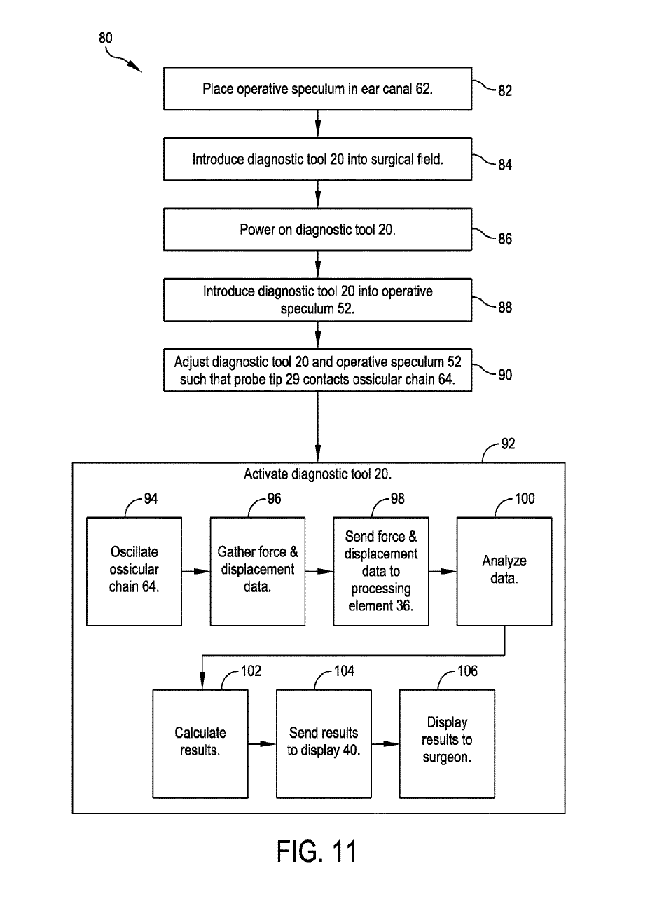

FIG. 11 provides a flow chart detailing an exemplary method 80 for using diagnostic tool 20 and operative speculum 52 in a surgical environment. Once the middle ear is properly exposed during the surgical procedure, one embodiment of method 80 initiates with the placement of operative speculum 52 (82). In this regard, operative speculum 52 is placed in ear canal 62 (FIG. 10) such that the surgeon has a proper view of the middle ear through the operative microscope (not shown), which may be aimed to look through operative speculum 52. Once speculum 52 is in position, diagnostic tool 20 may be brought into the surgical field (84) within sterile cover 44 and powered on (86). The surgeon may use one hand to stabilize operative speculum 52 and the other to introduce diagnostic tool 20 into operative speculum 52 (88) by advancing insert probe 24/sheath 46 through port 56 of operative speculum 52. Tool 20 and operative speculum 52 may then be adjusted (90) such that probe tip 29 is brought into contact with the desired portion of ossicular chain 64. This adjustment may be accomplished by adjusting tool 20 and operative speculum 52 independently or, in the case of a tapered port 56 of speculum 52, tool 20 may be locked in the engaged position (FIGS. 9 and 10) and manipulated as one unit. Once tip 29 is in contact with ossicular chain 64, tool 20 may be activated (92). Once tool 20 is activated, probe 24 begins to oscillate, which, in turn displaces/oscillates the bones of ossicular chain 64 (94). Method 80 continues when force and displacement data is gathered (96) and sent (98) to processing element 36. Once received by processing element 36, processing element 36 may analyze the data (100) and calculate (102) a number of results relating to the forces exerted by and the stiffness of the ossicular chain, as discussed above. These results are then sent (104) to display 40 and displayed (106) to the surgeon.

While method 80 is presented with respect to a surgical procedure, it may be equally applied to a conscious patient with an anesthetized eardrum. In the conscious situation, tool 20 may be placed within ear canal 62 of a patient in clinic and brought into contact with portions of the ear that are visible to the naked eye (e.g., the umbo, or a portion of the eardrum with an ossicle bone located behind it). Separate sets of normative data may be stored in processing element 36 for surgical and non-surgical analyses, taking into account the varying placements of diagnostic tool 20.

Using diagnostic tool 20 and a support and positioning device such as operative speculum 52, medical professionals may precisely quantify movement of the ossicular chain, thereby removing the subjectivity currently involved in diagnosing and treating conditions related to ossicular functionality and movement, such as otosclerosis. This ability leads to consistently accurate diagnoses, reduces the occurrence of unnecessary surgeries, and increases the success rate of the corrective surgeries performed.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A diagnostic tool for measuring an intraoperative stiffness of an ossicular chain of a human ear, comprising:
a chassis, said chassis housing a load cell that is operatively coupled to a linear actuator and a processing element; and
a rigid probe protruding from said chassis from a proximal end to a distal end, wherein said proximal end is operatively coupled to said load cell and said distal end is configured for placement against the ossicular chain of the human ear, wherein when said diagnostic tool is activated, said linear actuator displaces said load cell and said rigid probe through a predetermined distance in an oscillating manner, said load cell measures a force of said distal end of said rigid probe against the ossicular chain, and said processing element receives a data signal relating to said predetermined distance and said force and calculates a stiffness of the ossicular chain.

2. The diagnostic tool of claim 1, further comprising a display housed within said chassis and in communication with said processing element, wherein said display is configured to present data relating to said calculated stiffness of the ossicular chain to a user.

3. The diagnostic tool of claim 2, further comprising a battery housed within said chassis and operably coupled with said load cell, said linear actuator, said processing element, and said display.

4. The diagnostic tool of claim 1, wherein said diagnostic tool is a handheld device.

5. The diagnostic tool of claim 4, wherein said diagnostic tool is supported against the ossicular chain by a support and positioning structure.

6. The diagnostic tool of claim 5, wherein said support and positioning structure comprises an operative speculum adapted for placement in a canal of the ear, said operative speculum having an instrument port configured to receive and retain said rigid probe of said diagnostic tool in an engaged position in which said distal end of said rigid probe abuts the ossicular chain.

7. The diagnostic tool of claim 1, further comprising a sheath configured to envelop said rigid probe.

8. The diagnostic tool of claim 7, wherein said sheath is disposable.

9. The diagnostic tool of claim 7, wherein said sheath is sterilizable and reusable.

10. The diagnostic tool of claim 1, further comprising a cover configured to envelop said chassis of said diagnostic tool.

11. The diagnostic tool of claim 10, wherein said cover is disposable.

12. The diagnostic tool of claim 10, wherein said cover is sterilizable and reusable.

13. A system for analyzing the compliance of an ossicular chain of a human ear, comprising:
a diagnostic system, comprising:
a load cell coupled between a linear actuator and a rigid probe having a proximal end and a distal end adapted to abut the ossicular chain, wherein when activated, said linear actuator oscillates said load cell through a predetermined distance and said load cell measures a force exerted between said distal end of said rigid probe and said ossicular chain;
a processing element coupled with said load cell, said processing element having logic instructions configured to analyze displacement and force data from said diagnostic system and calculate a stiffness of the ossicular chain; and
a handheld chassis housing said load cell, said linear actuator, and said processing element, wherein said proximal end of said rigid probe protrudes from said handheld chassis.

14. The system of claim 13, further comprising an operative speculum comprising a cone-shaped body, said body configured to manipulate a canal of the ear and having an instrument port configured to receive and retain said rigid probe in an engaged position in which said distal end of said rigid probe abuts the ossicular chain.

15. The system of claim 14, wherein said handheld chassis further houses an analog-to-digital converter coupled between said processing element and a display, said display configured to provide a visual display of said calculated stiffness of the ossicular chain.

16. The system of claim 13, further comprising a hollow sheath configured to envelop said rigid probe.

17. The system of claim 13, further comprising a cover configured to envelop said handheld chassis.

18. The system of claim 13, wherein said distal end of said rigid probe is spherical, flattened, ridged, roughened, pointed, needled, concave, or u-shaped.

19. A method of measuring an intraoperative stiffness of an ossicular chain of a human ear using an operative speculum and a handheld ossicle-measurement tool, said operative speculum having an instrument port configured to align with the ossicular chain when said speculum is placed within an ear canal and said handheld ossicle-measurement tool having a load cell operatively coupled between a rigid probe and a linear actuator, said load cell and said linear actuator in communication with a processing element and a display, said method comprising:
placing said speculum within the ear canal such that said speculum provides visual access to the ossicular chain;
introducing said rigid probe of said handheld ossicle-measurement tool into said instrument port of said operative speculum such that a distal end of said probe contacts the ossicular chain; and
activating said ossicle-measurement tool such that (1) said linear actuator displaces said distal end of said rigid probe a predetermined distance against the ossicular chain, (2) said load cell measures a force of said rigid probe against the ossicular chain required to achieve movement of said predetermined distance, and (3) said processing element receives displacement and force data and calculates a stiffness of the ossicular chain.

20. The method of claim 19, further comprising said rigid probe comprising encasing within a disposable sheath.
